SEQUENCE LISTING (5 sequences)

SEQ ID NO: 1 - protein, 545 aa, Homo sapiens
MLLRSKPALPPPLLMLLLLGPLGPLSPGALPRPAQAQQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 2 - DNA, 1669 bp, Homo sapiens
atgctgctgcgctcgaagcctgcgctgccgccgccgctgctgatgctgctgctcctggggccgctgggtcccctctcccctggcgccctgccccgacctgcgcaagcacagcaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaatcaacctttgaagagagaagttactggcaatctcaagtcaaccaggatatttgcaaatatggatccatccctcctgatgtggaggagaagttacggttggaatggccctaccaggagcaattgctactccgagaacactaccagaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataacgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctgaaaataaaatatactagtcctgacactgaaaa

SEQ ID NO: 3 - protein, 592 aa, Homo sapiens [sequence omitted]

SEQ ID NO: 4 - DNA, 1927 bp, Homo sapiens
gcgagacccagtaggaagagaggtttaaatcagagggattgaatgagggtgctttgtgccttccctgaagccatgccctccagcaactcccgcccccccgcgtgcctagccccgggggctctctacttggctctgttgctccatctctccctttcctcccaggctggagacaggagacccttgcctgtagacagagctgcaggtttgaaggaaaagaccctgattctacttgatgtgagcaccaagaacccagtcaggacagtcaatgagaacttcctctctctgcagctggatccgtccatcattcatgatggctggctcgatttcctaagctccaagcgcttggtgaccctggcccggggactttcgcccgcctttctgcgcttcgggggcaaaaggaccgacttcctgcagttccagaacctgaggaacccggcgaaaagccgcgggggcccgggcccggattactatctcaaaaactatgaggatgacattgttcgaagtgatgttgccttagataaacagaaaggctgcaagattgcccagcaccctgatgttatgctggagctccaaagggagaaggcagctcagatgcatctggttcttctaaaggagcaattctccaatacttacagtaatctcatattaacagccaggtctctagacaaactttataactttgctgattgctctggactccacctgatatttgctctaaatgcactgcgtcgtaatcccaataactcctggaacagttctagtgccctgagtctgttgaagtacagcgccagcaaaaagtacaacatttcttgggaactgggtaatgagccaaataactatcggaccatgcatggccgggcagtaaatggcagccagttgggaaaggattacatccagctgaagagcctgttgcagcccatccggatttattccagagccagcttatatggccctaatattgggcggccgaggaagaatgtcatcgccctcctagatggattcatgaaggtggcaggaagtacagtagatgcagttacctggcaacattgctacattgatggccgggtggtcaaggtgatggacttcctgaaaactcgcctgttagacacactctctgaccagattaggaaaattcagaaagtggttaatacatacactccaggaaagaagatttggcttgaaggtgtggtgaccacctcagctggaggcacaaacaatctatccgattcctatgctgcaggattcttatggttgaacactttaggaatgctggccaatcagggcattgatgtcgtgatacggcactcattttttgaccatggatacaatcacctcgtggaccagaattttaacccattaccagactactggctctctctcctctacaagcgcctgatcggccccaaagtcttggctgtgcatgtggctgggctccagcggaagccacggcctggccgagtgatccgggacaaactaaggatttatgctcactgcacaaaccaccacaaccacaactacgttcgtgggtccattacactttttatcatcaacttgcatcgatcaagaaagaaaatcaagctgactgggactctcagagacaagctggttcaccagtacctgctgcagccctatgggcaggagggcctaaagtccaagtcagtgcaactgaatggccagcccttagtgatggtggacgacgggaccctcccagaattgaagccccgcccccttcgggccggccggacattggtcatccctccagtcaccatgggcttttttgtggtcaagaatgtcaatgctttggcctgccgctaccgataagctatcctcacactcatggctaccagtgggcctgctgggctgcttccactcctccactccagtagtatcctctgttttcagacatcctagcaaccagcccctgctg

SEQ ID NO: 5 - protein, 91 aa, Homo sapiens
METAVMNLAKGKEESLDSDLYAELRCLCIKTTSGIHPKNIQSLEVIGKGTHCNQVEVIATLKDGRKICLDPDAPRIKKIVQKKLAGDESAD

(19) United States
(12) Patent Application Publication
(10) Pub. No.: US 2015/0306185 A1
(43) Pub. Date: Oct. 29, 2015

(54) HEPARANASE AND ITS USES RELATED TO EXOSTOSES

(71) Applicant: UNIVERSITY OF ROCHESTER, Rochester, NY (US)

(72) Inventor: Matthew J. Hilton, Pittsford, NY (US)

(21) Appl. No.: 14/439,334

(22) PCT Filed: Nov. 1, 2013
(86) PCT No.: PCT/US2013/067997

§ 371 Date: Apr. 29, 2015

Related U.S. Application Data
(60) Provisional application No. 61/721,884, filed on Nov. 2, 2012.

(51) Int. Cl. A61K 38/47; A61K 49/00

(52) U.S. Cl. CPC A61K 38/47; A61K 49/0008; C12Y 302/01166

(57) ABSTRACT

Methods of treating or preventing exostosis in a subject comprising administering to a subject with exostosis or at risk of developing exostosis an effective amount of an agent that that cleaves heparan sulfate or an agent that increases expression of an enzyme that cleaves heparan sulfate are provided herein.

### CROSS-REFERENCE TO PRIORITY APPLICATION

This application claims priority to U.S. Provisional Application No. 61/721,884, filed Nov. 2, 2012, which is incorporated herein by reference in its entirety.

### STATEMENT REGARDING FEDERALLY FUNDED RESEARCH

This invention was made with government funding under NIH grant numbers AR057022, AR063071, AR059733, and AR061307. The government has certain rights in this invention.

### BACKGROUND

Development of exostosis during skeletal growth can lead to serious complications that include compression of soft tissue, nerves, and blood vessels and that result in severe pain and vascular complications. Additionally, abnormal endochondral bone growth and development of cartilagenous/bony exostosis interferes with normal weight bearing and joint mobility, which can result in long lasting and age-related joint problems. Due to these complications, individuals affected with osteochondroma-related conditions often undergo multiple surgeries to remove the exostoses. Unfortunately, following the surgical removal of exostosis during the skeletal growth phase, re-emergence can occur either due to incomplete removal of tissue or the development of a secondary exostosis. Thus, orthopedic surgery is an acceptable long-term or pain-free solution for treating these conditions.

### SUMMARY

Provided herein is a method of treating or preventing exostosis in a subject, comprising administering to a subject with exostosis or at risk of developing exostosis an effective amount of an agent that cleaves heparan sulfate.

Further provided is a method of treating or preventing an osteochondroma-related disease in a subject, comprising administering to a subject with or at risk of developing an osteochondroma-related condition, an effective amount of an agent that cleaves heparan sulfate.

Also, provided herein is a method of treating or preventing exostosis in a subject, comprising administering to a subject with exostosis or at risk of developing exostosis an effective amount of an agent that increases expression of an enzyme that cleaves heparan sulfate in the subject.

Further provided is a method of treating or preventing an osteochondroma-related disease in a subject, comprising administering to a subject with or at risk of developing an osteochondroma-related condition, an effective amount of an agent that increases expression of an enzyme that cleaves heparan sulfate in the subject.

Also provided is a method of identifying an agent that treats or prevents exostosis in a subject comprising contacting a transgenic animal comprising a deletion of both Ext1 alleles with a test agent such that if the test agent decreases or prevents exostosis in the animal, the test agent is an agent that treats or prevents exostosis in a subject.

### BRIEF DESCRIPTION OF THE FIGURES

FIG. 1 shows results obtained with a mouse model of MHE using the Col2CreERT2 and Ext1f/f mice combined with administration of low doses of tamoxifen to induce sporadic and clonal inactivation of both Ext1 alleles. The resulting cartilage specific, clustered deletions of the Ext1 alleles cause a localized disruption in heparan sulfate (HS) chain elongation on heparan sulfate proteoglycans (HSPGs) of mutant cells, while surrounding unaffected cells demonstrate normal HS biosynthesis. FIGS. 1 A1-A4 are photomicrographs showing that wild-type (WT) embryo sections normally exhibit a smooth border between the cartilage matrix and the surrounding perichondrium. FIGS. 1 B1-B4 are photomicrographs of tibia sections from Col2CreERT2; Ext1f/f mutant (MT) embryos at E18.5 demonstrating the early signs of exostosis formation near the perichondrial border of the cartilage growth plate. FIGS. 1A2-A4 show that the immature proliferating chondrocytes display normal organization into columnar structures that eventually undergo the process of hypertrophic differentiation. The hypertrophic chondrocytes enlarge, generate a mineralizing matrix, and secrete signaling molecules that promote the differentiation of perichondrial osteoblasts (FIG. 1 A4, white bar), which are responsible for laying down cortical bone matrix (FIG. 1 A4). As shown in FIG. 1A4, the perichondrial osteoblasts are cuboidal in shape and lay directly on the surface of their mineralizing matrix, while connective tissue and muscle cells have a more flattened appearance and flank the perichondrial osteoblast population. As shown in FIGS. 1 B2, E18.5 tibia sections from MT mice exhibit impaired proliferating, columnar chondrocyte structure, allowing for chondrocyte proliferation and expansion beyond the normal cartilage/perichondrial border (FIG. 1 B3, black arrow). These sections also demonstrate that exostosis formation developing near the hypertrophic chondrocyte and perichondrial border impairs normal osteoblast differentiation and bone formation. MT sections show a lack of mineralizing cortical bone matrix (FIG. 1 B4) combined with a loss of the cuboidal, perichondrial osteoblast population (FIG. 1 B4, no white bar), and the subsequent expansion of the flattened, connective tissue and muscle cell layers (FIG. 1 B4).

FIG. 2 shows photomicrographs of bone sections from two different genetic models. The left panel shows Matrilin1 Cre induces homogeneous recombination and deletion of floxed alleles throughout the cartilage of the distal femur, while the Col2Cre™ transgene induces sporadic (stochastic) recombination within the cartilage and surrounding perichondrial tissue in the distal femur (middle panel) and the proximal tibia (right panel).

FIG. 3 shows the cartilage phenotypes derived from homozygous and homogeneous deletion of Ext1 floxed alleles throughout the cartilage. Ext1^{Mat1} mutant embryos show an expansion of skeletal elements with longer proliferating and hypertrophic cartilage zones as compared to wildtype (WT). The set of two panels on the left shows photomicrographs of sections of an embryonic humerus (E17.5) at low power from mutant and wild type embryos and the set of two panels on the right shows the head of the humerus at higher magnification from the same sections.

FIG. 4 shows photomicrographs of a humeral section of a Ext1^{Mat1} mutant embryo (right panel) exhibiting a disorganized columnar zone of chondrocytes, which can often lead to cartilage malformations, and of wild-type (WT) embryonic humeral section (left panel). A disorganized columnar zone of chondrocytes often leads to cartilage malformations.

FIG. 5 shows photomicrographs of embryonic tibia (left set of panels) and radius (right et of panels) from Ext1^{Mat1} mutant (right) and wild type (left) at E17.5 days. Ext1^{Mat1} mutant embryos exhibit bowing of some long bones, which is often observed in Multiple Hereditary Exostoses (MHE). While complete deletion of Ext1 floxed alleles in cartilage can affect cartilage and bone development, exostoses (osteochondromas) rarely are found in these mutant mice.

FIG. 6 shows that stochastic deletion (˜50% of chondrocytes) of Ext1 floxed alleles using the Col2Cre™ transgene results in a delay in chondrocyte hypertrophy at embryonic day 14.5 (E14.5), which is represented by the smaller hypertrophic zone (arrow) in the humerus of a Ext1^{Mat1} mutant (right) as compared to an age-matched wild type embryo (left).

FIG. 7 shows that that stochastic deletion (˜50% of chondrocytes) of Ext1 floxed alleles using the Col2Cre™ transgene results in expansions of the cartilage regions, similar to the Ext1^{Mat1} mutant embryos. The set of two panels on the left shows photomicrographs of sections of an embryonic humerus (E17.5) at low power from mutant and wild type embryos and the set of two panels on the right shows the head of the humerus at higher magnification in the same sections.

FIG. 8 shows photomicrographs of a section of humerus of a Ext1^{C2TM} mutant (right) and wild type embryo (left) at 17.5 days. The Ext1^{C2TM} mutant embryos exhibit a disorganized columnar zone of chondrocytes, also similar to the Ext1^{Mat1} mutant embryos.

FIG. 9 shows photomicrographs of sections of tibia (left set of panels) and of radius (right set of panels) from a Ext1^{C2TM} mutant (right) and wild type embryo (left) at E17.5. The Ext1^{C2TM} mutant embryos display severe bowing of all mutant tibias and radius. This phenotype is much stronger and more penetrant in the stochastic deletion model.

FIG. 10 shows photomicrographs from various bone regions (diaphysis (left columns), proximal metaphysis (middle columns) and distal metaphysis (right columns) in the humerus of wild type (top) and Ext1^{C2TM} mutant (bottom) embryos (left) at 17.5 days. The photomicrographs are shown at low (left) and high magnification (right). Ext1^{C2TM} (stochastic deletion) mutant embryos develop numerous defects consistent with the development of exostoses or osteochondromas: presence of exostoses like cellular structures, persistent cartilage, and the presence of cartilage islands within the endocortical regions of the metaphysis. These phenotypes are either not observed or are dramatically reduced in Ext1^{Mat1} mutant embryos (complete and homogeneous Ext1 deletion in cartilage).

FIG. 11 shows that introduction of exogenous heparanase in all chondrocytes and surrounding cells corrects the delayed chondrocyte hypertrophy phenotype observed in Ext1^{C2TM} mutant embryos at E14.5. Photomicrographs of tibial sections from untreated wild-type (left), untreated Ext1^{C2TM} mutant (middle), and heparanase-treated mutant (right) embryos are shown.

FIG. 12 shows that introduction of exogenous heparanase in all chondrocytes and surrounding cells corrects the bowing phenotype observed in Ext1^{C2TM} mutant embryos at E17.5. Photomicrographs of sections from untreated wild-type (left), untreated Ext1^{C2TM} mutant (middle), and heparanase-treated mutants (right) are shown.

FIG. 13 demonstrates that introduction of exogenous heparanase corrects the expanded cartilage phenotype observed in Ext1^{C2TM} mutant embryos at E17.5 (arrows indicate length of marrow space, which is smaller when there is persistent cartilage). Photomicrographs of sections of humerus from untreated wild-type (left), untreated Ext1^{C2TM} mutant (middle), and heparanase-treated mutant (right) embryos are shown.

FIG. 14 shows that introduction of exogenous heparanase in all chondrocytes and surrounding cells corrects some of the disorganized columnar chondrocyte phenotype observed in Ext1^{C2TM} mutant embryos at E17.5. Photomicrographs of sections of humerus of columnar zones from untreated wild-type (left), untreated Ext1^{C2TM} mutant (middle), and heparanase-treated mutants (right) are shown.

### DETAILED DESCRIPTION

Provided herein is a method of treating or preventing exostosis in a subject. The method includes administering to a subject with exostosis or at risk of developing exostosis an effective amount of an agent that cleaves heparan sulfate.

As used throughout, exostosis or exostoses includes one or more occurrences of new bone formation on the surface of a bone. An exostosis is generally non-malignant but is an abnormal bone growth. Exostosis can occur in any bone where cartilage eventually forms bone, for example, in the long bones of the leg, the pelvis, or scapula, to name a few. Further, exostosis can be associated with hereditary and non-hereditary osteochondroma-related diseases such as osteochondromatosis and multiple hereditary exostoses (MHE), a disease in which multiple bony spurs or lumps develop on the bones of a subject. Multiple Hereditary Exostoses (MHE) is an autosomal dominant disorder characterized by short stature, bowing of bones, and multiple cartilage-capped bony exostoses (for example, a mass, a protuberance or benign osteochondromas) that develop near the growth plate region of endochondral or long bones. Other conditions associated with exostosis include, but are not limited to, subungual exostosis, buccal exostosis, torus mandibularis, and torus palatinus.

Further provided is a method of treating or preventing an osteochondroma-related disease in a subject. The method includes administering to a subject with or at risk of developing an osteochondroma-related condition, an effective amount of an agent that cleaves heparan sulfate.

As used throughout, by subject is meant an individual. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

Throughout this application, by treatment, treat or treating is meant a method of reducing or delaying one or more effects or symptoms of a disease. Treatment can also refer to a method of reducing the underlying pathology rather than just the symptoms. The treatment can be any reduction and can be, but is not limited to, the complete ablation of the disease or the symptoms of the disease. Treatment can also include the complete amelioration of a disease as detected by art-known techniques. Art recognized methods are available to detect osteochondroma-related conditions and their symptoms. These include, but are not limited to, ultrasonometric evaluation, bone density scan, radiological examination, histological examination, MRI, musculoskeletal evaluation and genetic analysis. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease (for example, a reduction in mass size or pain) in a subject as compared to a control. In the methods set forth herein, a control subject can be a subject that has not received an agent that cleaves heparan sulfate, for example, a heparanase, or an agent that increases expression of an enzyme that cleaves heparan sulfate, or the same subject prior to receiving an agent that cleaves heparan sulfate or an agent that increases expression of an enzyme that cleaves heparan sulfate.

As utilized herein, by reducing or preventing exostosis is meant delaying, averting, obviating, forestalling, stopping, or hindering the onset, incidence or severity of exostosis in a subject. For example, the disclosed method is considered to prevent exostosis if there is a reduction or delay in onset, incidence or severity of exostosis or one or more symptoms of exostosis as compared to a control subject. It is understood that the methods disclosed herein can be used to treat existing exostosis or prevent the formation of new exostosis.

Further provided is a method of treating or preventing exostosis in a subject, the method including administering to a subject with exostosis or at risk of developing exostosis an effective amount of an agent that increases expression of an enzyme that cleaves heparan sulfate in the subject

Also provided is a method of treating or preventing an osteochondroma-related disease in a subject, comprising administering to a subject with or at risk of developing an osteochondroma-related condition, an effective amount of an agent that increases expression of an enzyme that cleaves heparan sulfate.

In the methods set forth herein, an increase in expression can be an increase of about 10%, 20%, 30%, 40%, 50%, 60%, 70%, 80%, 90%, 100%, 150%, 200%, 250%, 300%, 350%, 400% or greater as compared to a control subject. Optionally, the increase in expression of an enzyme that cleaves heparan sulfate occurs in the cartilage cells of the subject. Since a subject can produce a basal level of one or more enzymes that cleave heparan sulfate, an increase can also be an increase of about 10%, 20%, 30%, 40%, 50%, 60%, 70%, 80%, 90%, 100%, 150%, 200%, 250%, 300%, 350%, 400% or greater as compared to the basal level of one or more enzymes in a subject.

The methods and compounds as described herein are useful for therapeutic treatment. Therapeutic treatment involves administering to a subject a therapeutically effective amount of one or more of the agents described herein, optionally, after diagnosis of exostosis or a disease associated with exostosis, for example, MHE. A subject with exostosis or at risk of developing exostosis can also be selected for treatment or prevention of exostosis.

In the methods provided herein, the agent that cleaves heparan sulfate can be any enzyme that cleaves heparan sulfate including, for example, a heparanase, such as heparanase 1, heparanase 2, heparanase III or CTAP-III. The heparanase can be from any species, including, but not limited to a human heparanase, a porcine heparanase, a bovine heparanase, an equine heparanase, a canine heparanase, an ovine heparanase, a rat heparanase or a bacterial heparanase, to name a few. Optionally, the enzyme can specifically cleave heparan sulfate without cleaving heparins, including low molecular weight heparins. Optionally, the enzyme cleaves heparan sulfate evenly throughout the subject's skeleton. As utilized throughout, a heparanase is an enzyme that acts at the cell surface and within the extracellular matrix to cleave polymeric heparan sulfate glycosaminoglycan molecules and degrade them into shorter chain length oligosaccharides. The heparanase can be chemically or recombinantly prepared. Heparanases can also be obtained commercially. For example, recombinant heparanase III, derived from Flavobacterium heparinum, can be obtained from IBEX Pharmaceuticals (Montreal, Canada). The amino acid sequence for human heparanase 1 (HSPE) and the nucleotide sequence encoding human heparanase 1 are provided herein as SEQ ID NO: 1 and SEQ ID NO: 2. Amino acids 1-35 of SEQ ID NO: 1 comprise a signal sequence. It is understood that any of the enzymes or fragments thereof provided herein can be administered with or without a signal sequence. For example, and not to be limiting, polypeptides of SEQ ID NO: 1 comprising amino acids 36-109 or amino acids 158-543 and fragments thereof can be administered, wherein the polypeptide is not the full-length heparanase encoded by SEQ ID NO: 1. The amino acid sequence for human heparanase 2 and the nucleotide sequence encoding human heparanase 2 are provided herein as SEQ ID NO: 3 and SEQ ID NO: 4. The amino acid sequence for human CTAP-III is provided herein as SEQ ID NO: 5. Amino acids 1-6 of SEQ ID NO: 5 comprise a signal sequence for SEQ ID NO: 5. Polypeptides of SEQ ID NO: 5 comprising amino acids 7-91 and fragments thereof can be administered, wherein the polypeptide is not the full-length CTAP-III encoded by SEQ ID NO: 5.

Modified heparanases can also be used in the methods set forth herein. For example, a polypeptide having at least, about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity to a wild type heparanase sequence set forth herein, wherein the polypeptide has heparanase activity, can also be used. Modified heparanases can have about 10%, 20%, 30%, 40%, 50%, 60%, 70%, 80%, 90% or 100% heparanase activity as compared to the wild-type enzyme. Modifications include, for example, insertions, substitutions and deletions. For example, a heparanase with one or more amino acid substitutions can also be used in the methods provided herein, so long as the heparanase function is maintained. Fragments of heparanases, including modified heparanases, can also be used as long as these fragments retain heparanase activity. A fragment can have about 10%, 20%, 30%, 40%, 50%, 60%, 70%, 80%, 90% or 100% heparanase activity as compared to the wild-type enzyme.

Nucleic acids encoding these sequences are also provided herein. These nucleic acids can be used for the recombinant production of the polypeptides set forth herein. Those of skill in the art readily understand how to determine the identity of two polypeptides or nucleic acids. For example, the identity can be calculated after aligning the two sequences so that the identity is at its highest level.

Another way of calculating identity can be performed by published algorithms. Optimal alignment of sequences for comparison can be conducted using the algorithm of Smith and Waterman Adv. Appl. Math. 2: 482 (1981), by the alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih.gov/blast/bl2seq/bl2.html), or by inspection.

The same types of identity can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 that are herein incorporated by this reference for at least material related to nucleic acid alignment. It is understood that any of the methods typically can be used and that, in certain instances, the results of these various methods may differ, but the skilled artisan understands if identity is found with at least one of these methods, the sequences would be said to have the stated identity.

For example, as used herein, a sequence recited as having a particular percent identity to another sequence refers to sequences that have the recited identity as calculated by any one or more of the calculation methods described above. For example, a first sequence has 80 percent identity, as defined herein, to a second sequence if the first sequence is calculated to have 80 percent identity to the second sequence using the Zuker calculation method even if the first sequence does not have 80 percent identity to the second sequence as calculated by any of the other calculation methods. As yet another example, a first sequence has 80 percent identity, as defined herein, to a second sequence if the first sequence is calculated to have 80 percent identity to the second sequence using each of calculation methods (although, in practice, the different calculation methods will often result in different calculated identity percentages).

Agents that increase expression of an enzyme that cleaves heparan sulfate in a subject include, but are not limited to, chemicals, small molecules, inorganic molecules, organic molecules, drugs, proteins, cDNAs, antibodies, morpholinos, triple helix molecules, peptides, siRNAs, shRNAs, miRNAs, antisense RNAs and ribozymes. One or more agents can be used to increase expression of one or more heparanases in vivo, ex vivo and in vitro. Optionally, one or more agents increase expression of one or more heparanases in cartilage cells, in vivo, ex vivo or in vitro. Optionally, one or more agents increase expression of one or more heparanases in cartilage cells evenly throughout the skeleton of a subject. By way of example, estrogen (17-beta-estradiol) and derivatives thereof, including tamoxifen (4-hydroxy-tamoxifen), can be used to increase endogenous expression of heparanases. See, for example, Xu et al. Human Reproduction 22(4): 927-937 (2007); Elkin et al. Cancer Research 63: 8821-8826 (2003); and Cohen et al. Clin. Cancer Res. 13: 4069-4077 (2007), incorporated herein by reference in their entireties.

One or more of the agents provided herein, for example, an agent that cleaves heparan sulfate and/or an agent that increases expression of an enzyme that cleaves heparan sulfate, can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of an agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy 22d edition Loyd V. Allen et al, editors, Pharmaceutical Press (2012). Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions described herein, suitable for parenteral injection, may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Administration can be carried out using therapeutically effective amounts of an enzyme that cleaves heparan sulfate or an agent that increases expression of an enzyme that cleaves heparan sulfate. For example, therapeutically effective amounts of heparanase or an agent that increases heparanase expression, can be used, for periods of time effective to treat or prevent exostosis or an osteochondroma-related disease in a subject. The effective amount may be determined by one of ordinary skill in the art and includes exemplary dosage amounts for a mammal of from about 0.5 to about 200 mg/kg of body weight per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day.

According to the methods taught herein, the subject is administered an effective amount of an enzyme that cleaves heparan sulfate, for example, heparanase, or an agent that increases expression of an enzyme that cleaves heparan sulfate. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Any appropriate route of administration may be employed, for example, parenteral, intravenous, subcutaneous, intramuscular, intraventricular, intracorporeal, intraperitoneal, rectal, or oral administration. Administration can be systemic or local. Pharmaceutical compositions can be delivered locally to the area in need of treatment, for example by topical application or local injection. Any of the compositions can be delivered via an implant that releases one or more of the compositions provided herein. For example, a microchip that is programmed to release one or more compositions can be implanted in the subject to deliver the composition to the bones of the subject and/or to the site of exostosis. Multiple administrations and/or dosages can also be used. Effective doses can be extrapolated from dose-response curves derived from in vitro or animal model test systems.

Multiple administrations and/or dosages can also be used. Effective doses can be extrapolated from dose-response curves derived from in vitro or animal model test systems.

Optionally, the agents described herein, are administered as a nucleic acid, for example, within a vector that encodes the agent. It is understood that, if, for example, a nucleic acid encoding heparanase is administered to a subject in a form that can be expressed within the subject, then heparanase has been administered to the subject.

In the methods described herein, which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the nucleic acid is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by gene gun or other delivery methods such as electroporation, the technology for which is available from Genetronics, Inc. (San Diego, Calif.) as well as by means of sonoporation.

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells a nucleic acid encoding, for example, heparanase. The exact method of introducing the nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

Any of the methods set forth herein can further comprise administering an anti-inflammatory agent to the subject. Examples of anti-inflammatory agents include, but are not limited to ImSAIDs, NSAIDS and steroids. Also, any of the methods set forth herein can further comprise the step of surgical removal of exostosis, either before or after administration of an enzyme that cleaves heparan sulfate or an agent that increases the expression of an enzyme that cleaves heparan sulfate in the subject.

Described herein is a mouse model that allows for cartilage specific, clonal deletions of both Ext1 alleles within the developing mouse. These animals develop a phenotype strikingly similar to human MHE. Exotoses are generated in this mouse model, not because HS (heparan sulfate) chain lengths are reduced in all cells, but because they are reduced only in sporadic, clusters of chondrocytes. It is likely that inappropriate signaling only occurs when the HS gradient is “uneven” within the cartilage, independent of the exact global level of HS synthesis or HS chain elongation. Therefore, this mouse model is not only useful for elucidating mechanisms of MHE pathogenesis but also for establishing therapeutic strategies that address a clinical problem that is currently only treated palliatively.

Since the generation of exostoses in humans and the mouse model set forth herein occur in a stochastic and largely unpredictable manner, a targeted therapy for MHE would be difficult to envision with current technologies. Therefore, a more universal approach at restoring an “even” HS and signaling gradient within the cartilage growth plate is necessary. For example, the use of heparanase, which is an enzyme that cleaves HS chains and is normally expressed in a variety of tissues is provided herein. The use of heparanase to globally reduce HS chain lengths to levels near that observed in Ext1 defective chondrocytes will not have a significant detrimental effect on overall skeletal development and growth. As disclosed herein, exostosis formation generated by clustered and sporadic chondrocyte specific deletions of both Ext1 alleles can be prevented by genetic activation or therapeutic administration of heparanase in order to globally reduce HS chain lengths, thereby creating a more uniform HS and signaling gradient within the cartilage extracellular matrix.

Thus provided is a transgenic animal comprising a deletion of one or both Ext1 alleles. Preferably the animal exhibits a sporadic or mosaic occurrence in deletion based on a lack of homogeneity in efficiency of the transgene. Optionally, the expression of the sequence used to knock-out or functionally delete one or both Ext1 alleles can be regulated by an appropriate promoter sequence. For example, constitutive promoters can be used to ensure that the functionally deleted gene is not expressed by the animal. In contrast, an inducible promoter can be used to control when the transgenic animal does or does not express the gene of interest. Exemplary inducible promoters include tissue-specific promoters and promoters responsive or unresponsive to a particular stimulus (such as light, oxygen, chemical concentration, such as a tetracycline or doxycycline inducible promoter). Exemplary transgenic non-human animals include, but are not limited to, ferrets, guinea piags, chinchilla, mice, monkeys, rabbits and rats. Progeny of these transgenic animals are also provided herein.

The transgenic animal can be an animal with homogeneous cartilage specific Ext1 deletions, or cartilage specific, clonal deletions of both Ext1 alleles as described in the Examples. As shown in the Examples, cartilage-specific, clustered deletions of the Ext1 alleles cause localized disruption in HS chain elongation on heparan sulfate proteoglycans (HSPGs) of mutant cells, while surrounding unaffected cells demonstrate normal HS biosynthesis. In order to obtain sporadic and clonal deletions of Ext1, the functional deletion of Ext1 can be controlled via Col2-Cre mediated deletion. As described in the Examples, the transgenic animal comprises a tamoxifen-inducible Cre transgene (Cre-ERT2) under the control of the Type II Collagen alpha (I) promoter. Optionally, the Cre transgene is inducible in a dose-responsive fashion to modulate the efficiency of Ext1 deletion in the transgenic animal. The transgenic animal also comprises a transgene comprising loxP flanked Ext1 alleles. Administration of tamoxifen at appropriate times induces Cre activity which effects sporadic and clonal inactivation of both Ext1 alleles.

Also provided is a method of identifying an agent that treats or prevents exostosis in a subject comprising contacting a transgenic animal comprising a functional deletion of both Ext1 alleles with a test agent such that if the test agent decreases or prevents exostosis in the animal, the test agent is an agent that treats or prevents exostosis in a subject.

Further provided is a method of identifying an agent that treats or prevents exostosis in a subject comprising contacting a Col2CreERT2; Ext1f/f transgenic animal with tamoxifen, and contacting the Col2CreERT2; Ext1f/f transgenic animal with a test agent such that if the test agent decreases or prevents exostosis in the transgenic animal, the test agent is an agent that treats or prevents exostosis in a subject. The Col2CreERT2; Ext1f/f transgenic animal can be contacted with the test agent before or after contacting the Col2CreERT2; Ext1f/f transgenic animal with tamoxifen to induce sporadic and clonal inactivation of both Ext1 alleles.

The disclosure also provides a pharmaceutical pack or kit comprising one or more containers filled with one or more of the ingredients of the pharmaceutical compositions. Instructions for use of the composition can also be included.

Agents identified using this method can be agents that cleave heparan sulfate. Agents identified using this method can be agents that increase expression of an enzyme that cleaves heparan sulfate in the transgenic animal. For example, the agent can increase heparanase expression in the cartilage cells of the transgenic animal. Optionally, the agent effects cleavage of heparan sulfate evenly throughout the skeleton of the transgenic animal, either directly or indirectly via increased expression of an enzyme that cleaves heparan sulfate. Agents identified using these methods can be used to treat or prevent exostosis or an osteochondroma-related disease in a subject.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Mice, Tamoxifen Injections and Administration of Heparanase III

Col2CreERT2 mice were generated previously by the methods set forth in Hilton et al. (Dev. Biol. 308(1):93-105 (2007)). Ext1f/f mice are from Dr. Yu Yamaguchi (Burnham Institute) and tg-hpa mice are from Dr. Israel Vlodaysky (Rappaport Institute). Col2CreERT2; Ext1f/f male mice were crossed with Ext1f/f; tghpa female mice to produce Col2CreERT2; Ext1f/f and Col2CreERT2; Ext1f/f; tg-hpa offspring in a 1:4 genotypic ratio. Ext1f/f and Ext1f/f; tg-hpa mice without the Cre recombinase were used as controls. Pregnant females from each cross were IP injected with low doses of tamoxifen at E13.5 in order to induce “clonal” deletion of the Ext1 alleles specifically within clusters of chondrocytes. Col2CreERT2; Ext1f/f male mice were crossed with Ext1f/f female mice to produce Col2CreERT2; Ext1f/f mutants and Ext1f/f controls. Similarly, gene deletion can be induced with low doses of tamoxifen at E13.5, although these animals will be treated daily with heparanase III (0.25-1.0 mg/kg/day) or vehicle from E13.5 until the time of tissue harvest (E18.5, 1 week, 1 month, and 4 months). The final time-point of 4 months was identified because it is a point at which the mouse skeleton has reached maturity. Concentrations of heparanase III doses can be altered for embryos and adult stages.

### Cartilage/Bone Histology and Histomorphometry

Embryonic and post-natal limbs at E17.5, 1 week, 1 month and 4 months were collected in ice cold sterile PBS, fixed in 10% buffered formalin overnight (E18.5 and 1 week) or for 3 days (1 and 3 months) at room temperature. The skeletal tissues were decalcified in 14% EDTA, pH 7.2 overnight (E18.5 and 1 week) or for 10 days (1 and 3 months) at room temperature. All tissues were then processed and embedded in paraffin prior to sectioning at 5-6 μm. Hemotoxylin and eosin and alcian blue/orange g staining was performed on tissue sections in order to analyze cellular distribution, tissue architecture, and cartilage/bone composition of developing limbs and exostoses from all mice. Histomorphometric measurements of cartilage/bone and exostoses were recorded using the Osteomeasure™ software on a microscope and Osteometrix™ imaging system (OsteoMetrics, Decatur, Ga.). The areas of alcian-blue stained cartilage and orange g stained bone were measured in growth plates, cortical bone regions, and exostoses for statistical analysis.

### In Situ Hybridization (ISH) and Immunohistochemistry (IHC)

ISH is performed on tissue sections using ^{35S}labeled riboprobes for chondrocyte (Col2a1, Agc1, Ihh, Col10a1, Mmp13) and osteoblast (Col1a1, Ap, Bsp, Oc) differentiation marker genes, regulatory molecules (Sox9, Runx2, Osx), and signaling pathway readouts (Wnt-Dkk1, Tcf1; FGF-Spry1, Spry2, Spry4; and Ihh-Patched1, Pthrp) using methods optimized previously (see, for example, Hilton et al. (Dev. Biol. 308(1):93-105 (2007)). The areas for each gene expression domain are quantified using Osteomeasure™ software. Chondrocyte proliferation assays are performed using BrdU immunostaining. For these assays, pregnant females are injected with BrdU at 0.1 mg/g body weight at 2 hours prior to harvest. BrdU detection is performed on paraffin sections using a kit from Invitrogen as previously described. IHC for native HS chains (10E4 antibody) are utilized to determine cartilage regions deficient in HS chains due to genetic recombination of Ext1 alleles and the in vivo administration of heparanase. Adjacent tissue sections are incubated with the heparanase antibody (mAb 130; InSight Ltd.) to detect transgenic and systemically administered heparanase.

Skeletal Staining.

Whole-mount alcian blue and alizarin red skeletal staining of embryos at E18.5 and post-natal pups at 1 week of age were performed. Cartilage (alcian blue) and bone (alizarin red) staining of the skeletons aid in identifying and calculating the number of exostoses formed on each element.

Micro-Computed Tomography (microCT) Imaging.

Prior to decalcification, both sets of hindlimbs from each animal harvested were subjected to high-resolution microCT scans to render 3D images of the hindlimb elements and the exostoses that develop (vivaCT 40; Scanco Medical AG). The two hindlimbs from each animal were scanned simultaneously in cone beam mode at 55 kEv, and an integration time of 300 milliseconds. Images from each hindlimb were reconstructed individually at identical thresholds to allow for comparing bone density and composition for each exostosis and the normal surrounding bone. The three-dimensional structure and histomorphometric analysis was performed using the Evaluation software of the iCT system. The bone volume per tissue volume (BV/TV) and cortical bone mineral density (Ct.BMD) was computed for each exostosis on all specimens from the 3D reconstructed image. Two-dimensional histomorphometric analysis was also performed on selected slices sampled from regions of interest including the exostosis and surrounding cortical bone. These 2D and 3D geometric and histomorphometric data from the microCT imaging provide robust quantitative information describing the volume and distribution of exostoses, as well as, the normal and aberrant bone formation observed from each animal. Data wereaveraged (+/−SEM) and ANOVA was performed to assess significant differences between groups (p value <0.05).

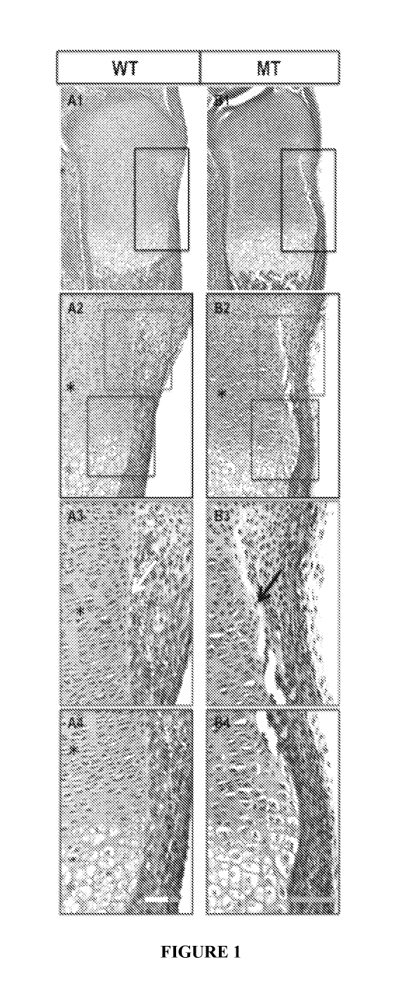

As set forth herein, a mouse model of MHE using the Col2CreERT2 and Ext1f/f mice combined with administration of low doses of tamoxifen to induce sporadic and clonal inactivation of both Ext1 alleles was made. The resulting cartilage specific, clustered deletions of the Ext1 alleles causeda localized disruption in HS chain elongation on HSPGs of mutant cells, while surrounding unaffected cells demonstrate normal HS biosynthesis. Histological analyses of tibia sections from Col2CreERT2; Ext1f/f mutant (MT) embryos at E18.5 demonstrated the early signs of exostosis formation near the perichondrial border of the cartilage growth plate (FIG. 1 B1-B4). Wild-type (WT) embryo sections normally exhibit a smooth border between the cartilage matrix and the surrounding perichondrium (FIG. 1 A1-A4, white arrow). The immature proliferating chondrocytes displayed normal organization into columnar structures (FIG. 1 A2-A4) that eventually undergo the process of hypertrophic differentiation (FIG. 1 A2, A4). The hypertrophic chondrocytes enlarged, generated a mineralizing matrix, and secreted signaling molecules that promote the differentiation of perichondrial osteoblasts (FIG. 1 A4, white bar), which are responsible for laying down cortical bone matrix (FIG. 1 A4). The perichondrial osteoblasts were cuboidal in shape and lay directly on the surface of their mineralizing matrix, while connective tissue and muscle cells had a more flattened appearance and flanked the perichondrial osteoblast population (FIG. 1 A4). E18.5 tibia sections from our MT mice exhibited impaired proliferating, columnar chondrocyte structure (FIG. 1 B2), allowing for chondrocyte proliferation and expansion beyond the normal cartilage/perichondrial border (FIG. 1 B3, black arrow). These sections also demonstrated that exostosis formation developing near the hypertrophic chondrocyte and perichondrial border impaired normal osteoblast differentiation and bone formation. MT sections showed a lack of mineralizing cortical bone matrix (FIG. 1 B4, no pink bone matrix) combined with a loss of the cuboidal, perichondrial osteoblast population (FIG. 1 B4, no white bar), and the subsequent expansion of the flattened, connective tissue and muscle cell layers (FIG. 1 B4). These data demonstrated the reliability of the MHE mouse model provided herein and validated the “clonal” loss-of-heterozygosity hypothesis, which allows development of therapeutic interventions for MHE.

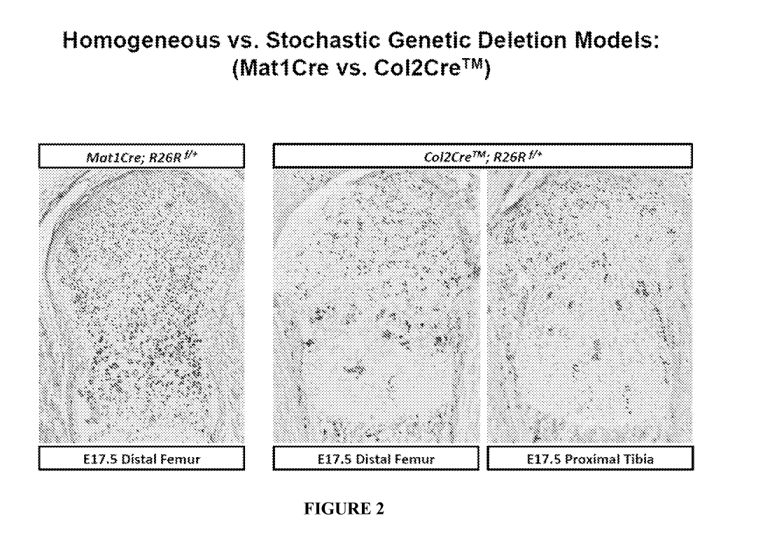

As shown in FIG. 2, while another model, the Matrilin1 Cre (Ext1^{Mat1}) model, induced homogenous recombination and deletion of floxed alleles throughout the cartilage, the Col2Cre™ transgene described herein induced sporadic (stochastic) recombination within the cartilage and surrounding perichondrial tissue. This sporadic, clonal deletion allowed for a phenotype that is consistent with what is observed in subjects with exostoses.

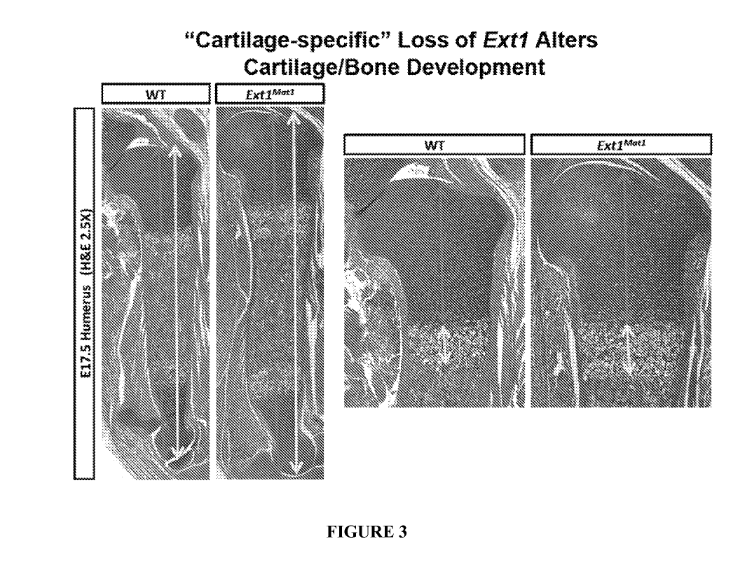

Table 1 shows that cartilage-specific Loss of Ext1 results in excessive cartilage in Ext1^{Mat1} mice. FIG. 3 shows the cartilage phenotypes derived from homozygous and homogeneous deletion of Ext1 floxed alleles throughout the cartilage. Ext1^{Mat1} mutant embryos showed an expansion of skeletal elements with longer proliferating and hypertrophic cartilage zones.

TABLE 1
Cartilage-specific Loss of Ext1 Results in Excessive Cartilage

| | Total Humerus Cartilage * |
|---|---|
| WT | 56% |
| Ext1^{Mat1} | 61% |
| | Total Tibial Cartilage * |
| WT | 53% |
| Ext1^{Mat1} | 57% |

N ≧ 3
* statistical significance p < 0.05

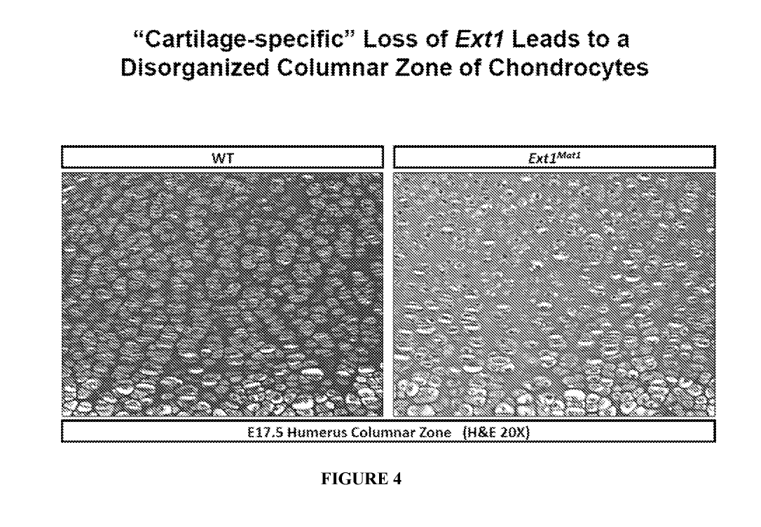

FIG. 4 shows that the Ext1^{Mat1} mutant embryos exhibited a disorganized columnar zone of chondrocytes, which can often lead to cartilage malformations.

Table 2 shows that cartilage-specific loss of Ext1 resulted in bowing of some bones.

TABLE 2
Cartilage-specific Loss of Ext1 Results in Bowing of Some Bones -

| | Bowing of | Bowing of | Bowing of |
|---|---|---|---|
| | the Tibia | the Radius | the Ulna |
| WT | 0% | 0% | 0% |
| Ext1^{Mat1} | 90% | 33.3% | 0% |

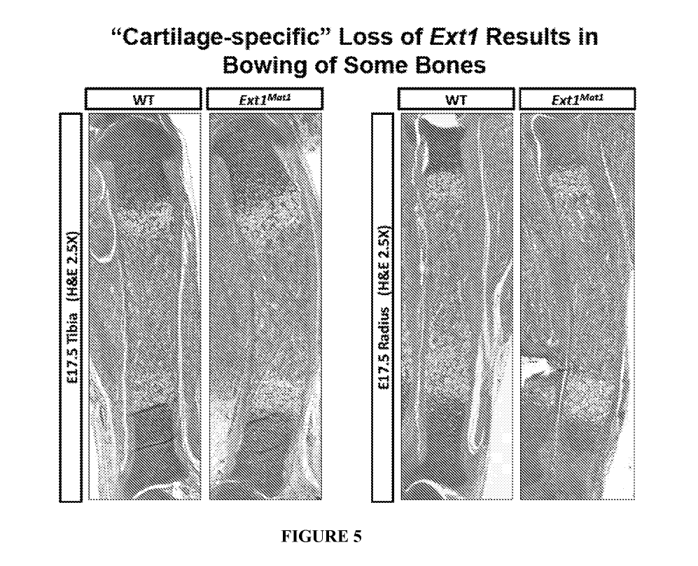

FIG. 5 illustrates that Ext1^{Mat1} mutant embryos exhibited bowing of some long bones, which is often observed in Multiple Hereditary Exostoses (MHE). While complete deletion of Ext1 floxed alleles in cartilage can affect cartilage and bone development, exostoses (osteochondromas) rarely are found in these mutant mice.

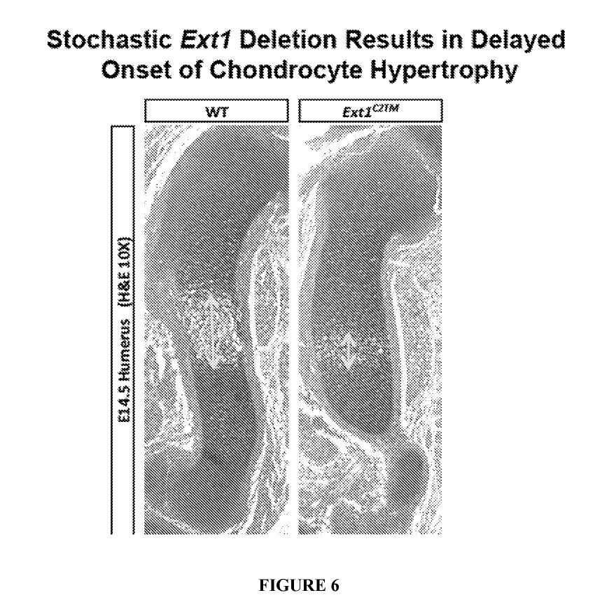

FIG. 6 shows that the animal model provided herein, i.e., stochastic deletion (˜50% of chondrocytes) of Ext1 floxed alleles using the Col2Cre™ transgene resulted in a delay in chondrocyte hypertrophy at embryonic day 14.5 (E14.5), which is represented by the smaller hypertrophic zone (arrow).

Table 3 shows that stochastic Ext1 deletion resulted in excessive cartilage

TABLE 3
Stochastic Ext1 deletion results in excessive cartilage.

| | Total Humerus Cartilage * |
|---|---|
| WT | 55% |
| Ext1^{C2TM} | 69% |
| | Total Tibial Cartilage * |
| WT | 59% |
| Ext1^{C2TM} | 62% |

N ≧ 3
* statistical significance p < 0.05

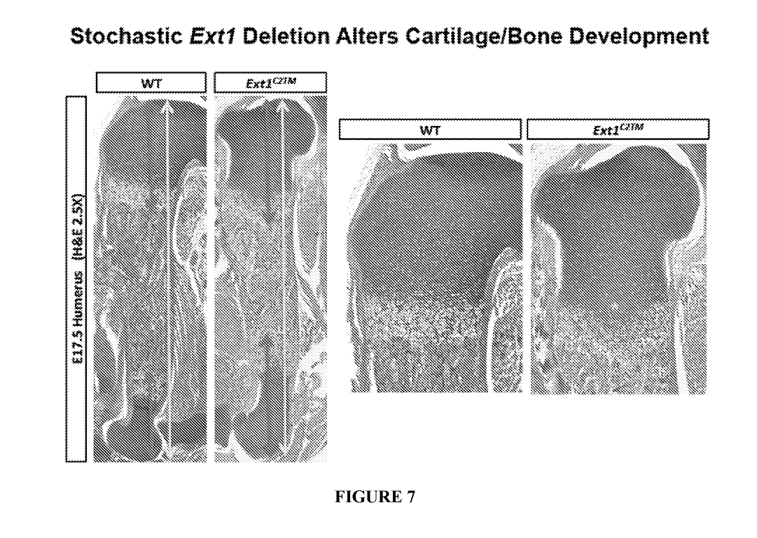

FIG. 7 shows that that stochastic deletion (˜50% of chondrocytes) of Ext1 floxed alleles using the Col2Cre™ transgene resulted in expansions of the cartilage regions, similar to the Ext1^{Mat1} mutant embryos.

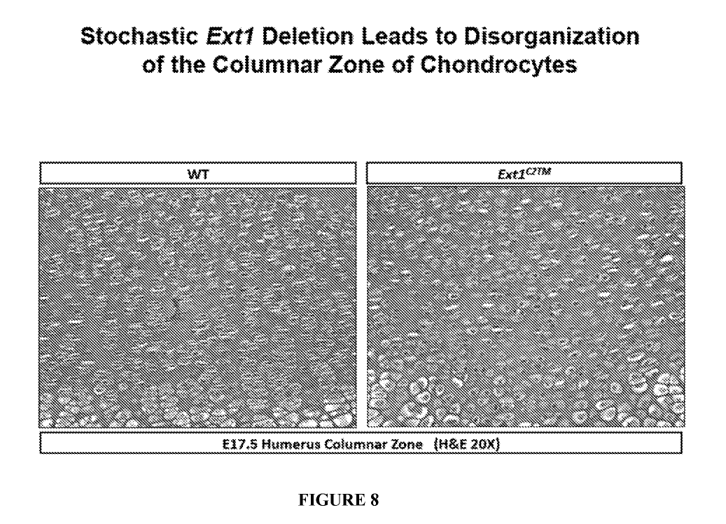

FIG. 8 shows that the Ext1^{C2TM} mutant embryos exhibited a disorganized columnar zone of chondrocytes, also similar to the Ext1^{Mat1} mutant embryos.

Table 4 shows that stochastic deletion resulted in bowing of some bones.

TABLE 4
Stochastic Ext1 deletion results in bowing of some bones

| | Bowing of | Bowing of | Bowing of |
|---|---|---|---|
| | the Tibia | the Radius | the Ulna |
| WT | 0% | 0% | 0% |
| Ext1^{C2TM} | 100% | 100% | 0% |

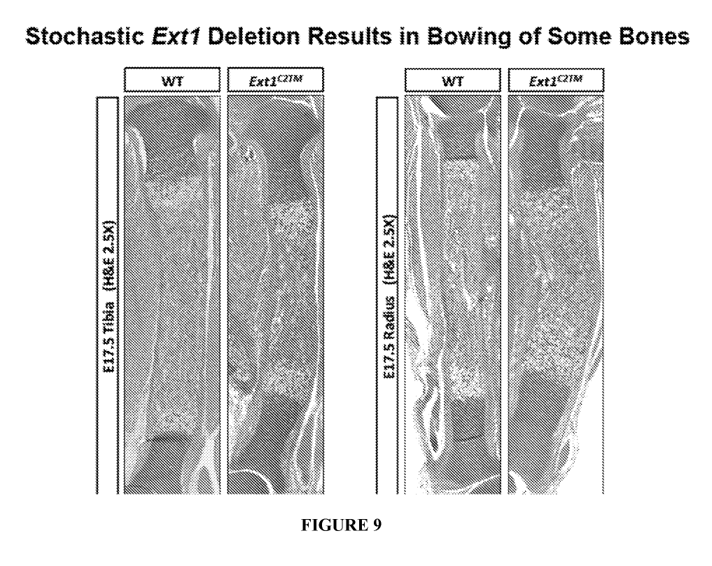

FIG. 9 shows that Ext1^{C2TM} mutant embryos displayed severe bowing of all mutant tibias and radius. This phenotype was much stronger and more penetrant in the stochastic deletion model.

Table 5 shows a comparison of how Ext1 efficiency affects the penetrance of MHE-related phenotypes in Ext1^{C2TM} and Ext1^{Mat1} embryos.

TABLE 5
Ext1 Deletion Efficiency Affects the Penetrance of MHE-Related
Phenotypes

| | | | Presence of | | | |
|---|---|---|---|---|---|---|
| | Presence of | | Persistent | | Presence of | |
| | Exostoses | | Cartilage | | Cartilage Islands | |
| | FL | HL | FL | HL | FL | HL |
| WT | 0% | 0% | 0% | 0% | 0% | 0% |
| Ext1^{C2TM} | 37.5% | 44.4% | 87.5% | 100% | 75% | 22.2% |
| WT | 0% | 0% | 0% | 0% | 0% | 0% |
| Ext1^{Mat1} | 11.1% | 10.0% | 55.6% | 30% | 0% | 0% |

N ≧ 8

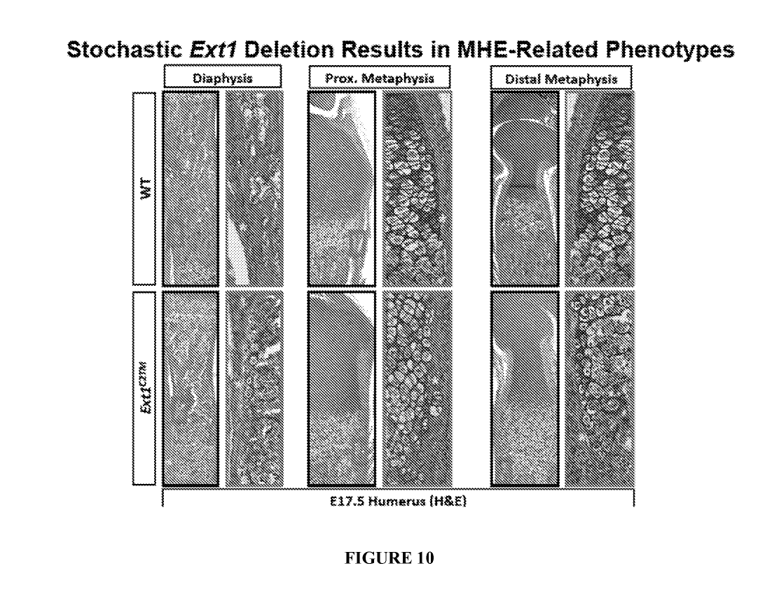

FIG. 10 shows that Ext1^{C2TM} (stochastic deletion) mutant embryos developed numerous defects consistent with the development of exostoses or osteochondromas: presence of exostoses like cellular structures, persistent cartilage, and the presence of cartilage islands within the endocortical regions of the metaphysis. These phenotypes were either not observed or were dramatically reduced in Ext1^{Mat1} mutant embryos (complete and homogeneous Ext1 deletion in cartilage).

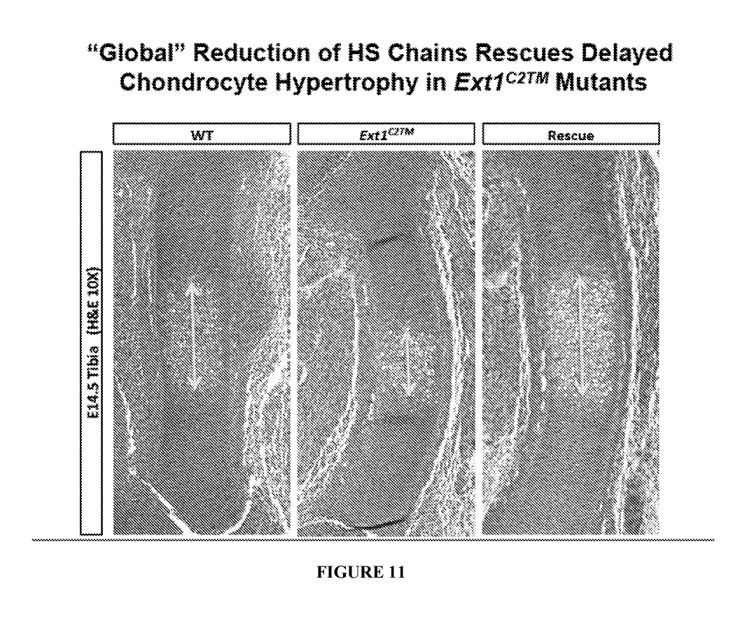

FIG. 11 shows that introduction of exogenous heparanase in all chondrocytes and surrounding cells corrected the delayed chondrocyte hypertrophy phenotype observed in Ext1^{C2TM} mutant embryos at E14.5.

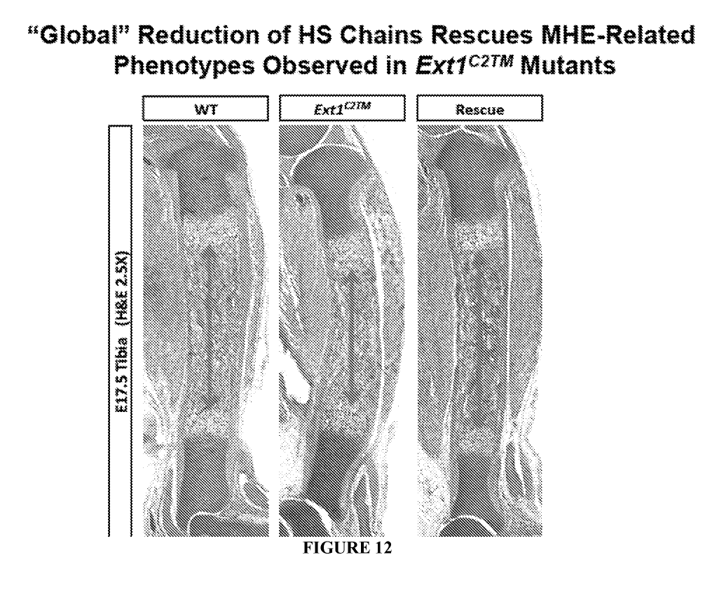

FIG. 12 shows that introduction of exogenous heparanase in all chondrocytes and surrounding cells corrected the bowing phenotype observed in Ext1^{C2TM} mutant embryos at E17.5. Exogenous heparanase was introduced via a transgene that encodes human heparanase I (HSPE).

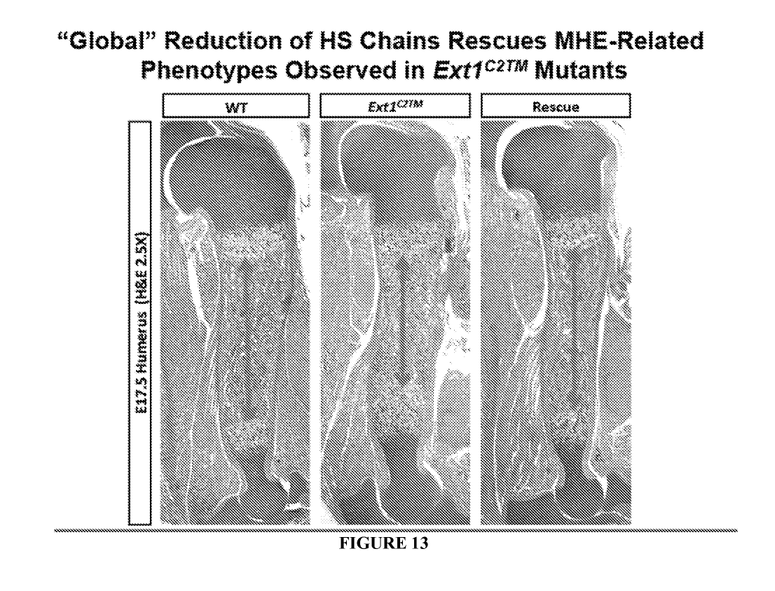

FIG. 13 demonstrates that introduction of exogenous heparanase in all chondrocytes and surrounding cells corrected the expanded cartilage phenotype observed in Ext1^{C2TM} mutant embryos at E17.5 (arrows indicate length of marrow space, which is smaller when there is persistent cartilage).

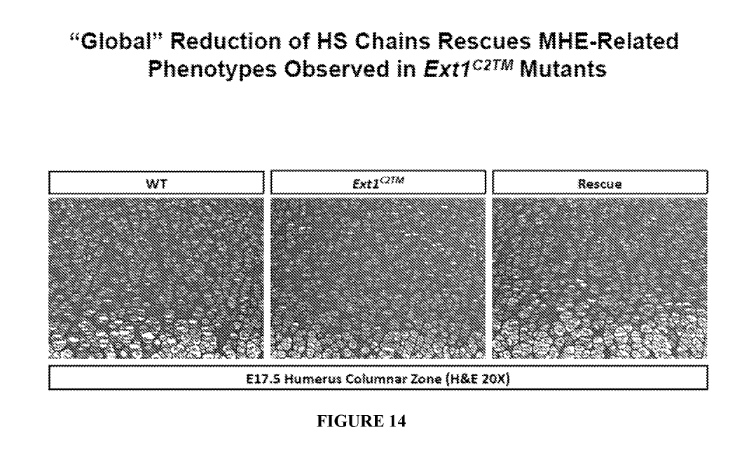

FIG. 14 shows that introduction of exogenous heparanase in all chondrocytes and surrounding cells corrected some of the disorganized columnar chondrocyte phenotype observed in Ext1^{C2TM} mutant embryos at E17.5.

Thus, overexpression of heparanase in the Ext1^{C2TM} animal model described herein, corrected MHE related phenotypes. Thus, provided herein is a new animal model of MHE that can be used to study the pathogenesis of exostosis formation and to evaluate the efficacy of therapeutics aimed at treating or preventing the disease.

1. A method of treating or preventing exostosis in a subject, comprising administering to a subject with exostosis or at risk of developing exostosis an effective amount of an agent that cleaves heparan sulfate.

2. The method of claim 1, wherein the agent is a heparanase.

3. A method of treating or preventing an osteochondroma-related disease in a subject, comprising administering to a subject with or at risk of developing an osteochondroma-related condition, an effective amount of an agent that cleaves heparan sulfate.

4. The method of claim 3, wherein the agent is a heparanase.

5. The method of claim 3, wherein the disease is multiple hereditary exostoses (MHE).

6. A method of treating or preventing exostosis in a subject, comprising administering to a subject with exostosis or at risk of developing exostosis an effective amount of an agent that increases expression of an enzyme that cleaves heparan sulfate in the subject.

7. The method of claim 6, wherein the enzyme is a heparanase.

8. A method of treating or preventing an osteochondroma-related disease in a subject, comprising administering to a subject with or at risk of developing an osteochondroma-related condition, an effective amount of an agent that increases expression of an enzyme that cleaves heparan sulfate in the subject.

9. The method of claim 8, wherein the enzyme is a heparanase.

10. The method of claim 7, wherein the disease is multiple hereditary exostoses (MHE).

11. A method of identifying an agent that treats or prevents exostosis in a subject comprising contacting a transgenic animal comprising a functional deletion of one or both Ext1 alleles with a test agent, wherein if the test agent decreases or prevents exostosis in the animal, the test agent is an agent that treats or prevents exostosis in a subject.

12. The method of claim 11, wherein both Ext1 alleles are functionally deleted.

13. The method of claim 11, wherein the transgenic animal comprises a tamoxifen-inducible Cre transgene (Cre-ERT2) under the control of the Colalpha(II) promoter. and a transgene comprising loxP flanked Ext1 alleles.
